(19) United States
(12) Patent Application Publication
(10) Pub. No.: US 2004/0127563 A1
(43) Pub. Date: Jul. 1, 2004

(54) Methods of performing medical procedures which promote bone growth, compositions which promote bone growth, and methods of making such compositions

(76) Inventor: Richard J. Deslauriers, Woodbury, CT (US)

Correspondence Address: BAKER BOTTS, LLP, 910 LOUISIANA, HOUSTON, TX, 77002-4995, US

(21) Appl. No.: 10/395,001

(22) Filed: Mar. 24, 2003

Related U.S. Application Data
(60) Provisional application No. 60/366,335, filed on Mar. 22, 2002.

(51) Int. Cl. A61K 31/225; C12N 5/08

(52) U.S. Cl. 514/547; 435/366

(57) ABSTRACT

In an exemplary embodiment of the present invention, a method of performing a medical procedure includes the step of applying a particular material to a portion of a bone of a mammal. The particular material includes at least one glyceride and stimulates bone growth. For example, cyclic adenosine monophosphate regulated lipases may metabolize the particular material after the particular material is applied to the bone. The at least one glyceride of the particular material and water within the mammal then may be converted into glycerol, fatty acid, and protons, which stimulates a conversion of osteoprogenitor cells into osteoblast cells. Subsequently, the glycerol and the fatty acid may be oxidized, and the oxidation may stimulate the conversion of adenosine diphosphate within the mammal into adenosine triphosphate. The osteoblast cells then may convert the adenosine triphosphate into adenosine diphosphate, such that energy is released. Moreover, the osteoblast cells use the energy to synthesize at least Type-I collagen proteins within the mammal, which creates the bone growth.

The present application claims priority from U.S. Provisional Patent Application No. 60/366,335, entitled “three part biodegradable osteoconductive polymer and dispensing system,” and filed on Mar. 22, 2002, the disclosure of which is incorporated herein by reference in its entirety.

### BACKGROUND OF THE INVENTION

1. Field of the Invention

The present invention relates generally to methods of performing medical procedures. In particular, the present invention is directed toward methods of performing medical procedures in which a particular material including one or more glyceride is used to promote bone growth.

2. Description of Related Art

Human bone includes a solid mineral phase and an organic matrix which is between 90% and 95% type I collagen. The mineral phase includes calcium and phosphate. Osteoblast cells synthesize and secrete the organic matrix, and mineralization of the organic matrix begins after the osteoblast cells secrete the organic matrix. Nevertheless, such mineralization of the organic matrix takes several weeks to complete. As the osteoblast cells secrete the organic matrix, the matrix surrounds the osteoblast cells, such that the osteoblast cells are transformed into osteocyte cells. Resorption of the bone is carried out mainly by osteocyte cells. Osteoclast cells are multinucleated cells formed by fusion of precursor hematopoietic stem cells.

Functions performed by bone are classified as mechanical, mineral storage, and hemopoietic. The mechanical properties of bone are related to its specific type of construction and internal architecture. Although bone may be relatively light, bone also may have a relatively high tensile strength. This combination of strength and weight results from the hollow, tubular shape of bone, the layering of bone tissue, and the internal buttressing of the organic matrix. Intramembranous ossification refers to the mechanism by which bone tissue supplants membranous or fibrous tissue. Bone tissue only grows by appositional growth, i.e., the deposition of a new organic matrix on the surface of the bone by adjacent surface cells. The repair of bone occurs in three phases starting with the inflammatory phase. During this phase, there is extensive tearing of the periosteum. The resultant rupture of blood vessels and extensive hemorrhage leads to a secondary inflammatory response of polymorphonuclear leukocytes, macrophages, and mononuclear cells. Pluripotential mesenchymal cells from the soft tissue and within the bone marrow give rise to the osteoblast cells that synthesize bone.

In 1849, it was discovered that an isocyanate group reacts with a hydroxyl group to generate a urethane, and in the 1940s it was discovered that a polyurethane polymer having two or more isocyanate groups per molecule reacts with a polyol or polyfunctional alcohol. It is known that naturally occurring fatty acids can be treated as natural polyols. Naturally occurring fatty acids obtained by means of hydrolysis of vegetable oils or similar methods may be esterified with glycols producing two or more free hydroxyl functional groups in a chain. In particular, it is know that castor oil possesses a high concentration of 12-hydroxylic acid triglycerol and can be treated as a natural polyol because the triglycerol molecule contains three hydroxyl side groups. Using a pre-polymerization process, the molecular weight can be increased, such that the polymerized unit has the structure similar to that of a naturally occurring triglycerol. Further this triglycerol matrix has specific mechanical properties. Minerals can be added to this triglycerol matrix, such that pH and mechanical properties can be controlled. Further the pre-polymerization process acts to release energy, such that the reaction temperature and the resulting exothermic output can be controlled and an minimized.

The initial utilization of fat as an energy source is the hydrolysis of triacylglycerol by lipases. The activity of cell lipase is regulated by hormones. Epinephrine, norepinephrine, glucagons, and adrenocorticotropic hormones stimulate the adenylate cyclase of cells. The increased level of cyclic adenosine monophosphate (cyclic AMP) then stimulates a protein kinase, which activates the lipase by phosphorylating the lipase. Thus, epinephrine, norepinephrine, glucagons, and adrenocorticotropic hormones cause lipolysis. glycerol formed by lipolysis is phosphorylated and oxidized to dihydroxyacetone phosphate, which in turn is isomerized to glyceraldehydes 3-phosphate. This intermediate is on both the glycolytic and the gluconeogenic pathways.

Known bone replacement technologies can be divided into three transitional matrix categories. The first category relies on replacing bone with either autogenous, homologous, heterologous, or decalcified bone and subsequent remodeling. The second category relies on replacing bone with a bone like mineral such as crystalline hydroxyapatite or calcium pyrophosphate or similar and subsequent remodeling. The third category relies on replacing bone with a material that maintains its chemical and mechanical properties without change (Titanium, Stainless Steel, PMMA), i.e., without subsequent remodeling. Nevertheless, these known methods do not allow for the growth of new bone.

### SUMMARY OF THE INVENTION

Therefore, a need has arisen for methods of performing medical procedures which overcome these and other shortcomings of the related art. A technical advantage of the present invention is that a material comprising at least one glyceride may be positioned in the vicinity of a bone of a mammal, e.g., in the vicinity of a damaged portion of the bone, and the material promotes bone growth. For example, the material can be applied to an exterior surface of the bone, dispensed in an opening formed within or through the bone, injected into the bone, positioned between two pieces of bone, or the like, without having to expose the bone, e.g., by injecting the material through the skin using a syringe. The material also may be molded into an implant, a screw, a plate, a prosthetic member, or the like, which may be inserted in or positioned on the bone. The material initially may be liquid, and then may cure into a sold. For example, the material may cure into a solid in an oxygen environment and/or a hydrophilic environment. The material may be used to reconstruct bone, fuse bones (intravertebroinfusions), reduce or eliminate bone fractures or otherwise damages bones, and/or regenerate missing bone, e.g., generate bone to fill a void within the bone. The material also may be used to make plates, screws, prosthetic joints, or the like, and/or may act as an anchor for a suture inserted in an opening in the bone, such that the suture does not fall out of the opening after insertion. Moreover, the material may be used as a base of a substrate in order to dilate compressed structures, e.g., vertebral disks, intramedullary nails, and in angioplasty type procedures. The internal rigid fixation of the newly generated bone is about the same as bone already present in the body, such that the generated bone is not readily damaged. Moreover, a first portion of the material may be contained in a first syringe and a second portion of the material may be packaged in a second syringe. The first and the second syringe may be packaged together in a moisture resistant package, such that the first portion of the material and the second portion of the material may be maintained in the same package but separate from each other. The first portion of the material and the second portion of the material may be readily combined to form the material when it is time to perform the medical procedure by dispensing the first portion and the second portion from the first syringe and the second syringe, respectively.

According to an embodiment of the present invention, a method of performing a medical procedure comprises the step of applying a particular material to a portion of a bone of a mammal. The particular material comprises at least one glyceride and stimulates bone growth. For example, cyclic adenosine monophosphate regulated lipases may metabolize the particular material after the particular material is applied to the bone. The at least one glyceride of the particular material and water within the mammal then may be converted into glycerol, fatty acid, and protons, which stimulates a conversion of at least one osteoprogenitor cell into an osteoblast cell. Subsequently, the glycerol or the fatty acid, or both, may be oxidized, and the oxidation may stimulate the conversion of adenosine diphosphate within the mammal into adenosine triphosphate. The osteoblast cells then may convert the adenosine triphosphate into adenosine diphosphate, such that energy is released. Moreover, the osteoblast cells use the energy to synthesize at least Type-I collagen proteins within the mammal, which creates the bone growth.

According to another embodiment of the present invention, a method of performing a medical procedure comprises the step of dispensing a particular material into an opening formed within or through at least one portion of a bone of a mammal. The particular material comprises at least one glyceride and stimulates bone growth. For example, cyclic adenosine monophosphate regulated lipases may metabolize the particular material after the particular material is dispensed within the opening. The at least one glyceride of the particular material and water within the mammal then may be converted into glycerol, fatty acid, and protons, which stimulates a conversion of at least one osteoprogenitor cell into an osteoblast cell. Subsequently, the glycerol or the fatty acid, or both, may be oxidized, and the oxidation may stimulate the conversion of adenosine diphosphate within the mammal into adenosine triphosphate. The osteoblast cells then may convert the adenosine triphosphate into adenosine diphosphate, such that energy is released. Moreover, the osteoblast cells use the energy to synthesize at least Type-I collagen proteins within the mammal, which creates the bone growth.

According to yet another embodiment of the present invention, a method of performing a medical procedure comprises the steps of forming a mold, and dispensing a liquid, particular material into the mold. The particular material solidifies within the mold and comprises at least one glyceride. The method also comprises the steps of removing the solidified, particular material from the mold, and implanting the solidified, particular material into an opening formed within or through at least one portion of a bone of a mammal. Moreover, the implanted, particular material stimulates bone growth. For example, cyclic adenosine monophosphate regulated lipases may metabolize the solidified, particular material after the solidified, particular material is implanted within the opening. The at least one glyceride of the solidified, particular material and water within the mammal then may be converted into glycerol, fatty acid, and protons, which stimulates a conversion of at least one osteoprogenitor cell into an osteoblast cell. Subsequently, the glycerol or the fatty acid, or both, may be oxidized, and the oxidation may stimulate the conversion of adenosine diphosphate within the mammal into adenosine triphosphate. The osteoblast cells then may convert the adenosine triphosphate into adenosine diphosphate, such that energy is released. Moreover, the osteoblast cells use the energy to synthesize at least Type-I collagen proteins within the mammal, which creates the bone growth.

According to still yet another embodiment of the present invention, a method of performing a medical procedure comprises the steps of forming a mold, and dispensing a liquid, particular material into the mold. The particular material solidifies within the mold and comprises at least one glyceride. The method also comprises the steps of removing the solidified, particular material from the mold, and positioning the solidified, particular material on at least one portion of a bone of a mammal. Moreover, the positioned, particular material stimulates bone growth. For example, cyclic adenosine monophosphate regulated lipases may metabolize the solidified, particular material after the solidified, particular material is positioned on the bone. The at least one glyceride of the solidified, particular material and water within the mammal then may be converted into glycerol, fatty acid, and protons, which stimulates a conversion of at least one osteoprogenitor cell into an osteoblast cell. Subsequently, the glycerol or the fatty acid, or both, may be oxidized, and the oxidation may stimulate the conversion of adenosine diphosphate within the mammal into adenosine triphosphate. The osteoblast cells then may convert the adenosine triphosphate into adenosine diphosphate, such that energy is released. Moreover, the osteoblast cells use the energy to synthesize at least Type-I collagen proteins within the mammal, which creates the bone growth.

According to another embodiment of the present invention, a method of performing a medical procedure comprises the step of injecting a particular material into a bone of a mammal. The particular material comprises at least one glyceride and stimulates bone growth within the bone. For example, cyclic adenosine monophosphate regulated lipases may metabolize the particular material after the particular material is injected into the bone. The at least one glyceride of the particular material and water within the mammal then may be converted into glycerol, fatty acid, and protons, which stimulates a conversion of at least one osteoprogenitor cell into an osteoblast cell. Subsequently, the glycerol or the fatty acid, or both, may be oxidized, and the oxidation may stimulate the conversion of adenosine diphosphate within the mammal into adenosine triphosphate. The osteoblast cells then may convert the adenosine triphosphate into adenosine diphosphate, such that energy is released. Moreover, the osteoblast cells use the energy to synthesize at least Type-I collagen proteins within the mammal, which creates the bone growth.

According to yet another embodiment of the present invention, a method of performing a medical procedure comprises the step of positioning a particular material between a first bone portion of a mammal and a second bone portion of the mammal. The particular material comprises at least one glyceride and stimulates a growth of a third bone portion which fuses the first bone portion to the second bone portion. For example, cyclic adenosine monophosphate regulated lipases may metabolize the particular material after the particular material is positioned between the first bone portion and the second bone portion. The at least one glyceride of the particular material and water within the mammal then may be converted into glycerol, fatty acid, and protons, which stimulates a conversion of at least one osteoprogenitor cell into an osteoblast cell. Subsequently, the glycerol or the fatty acid, or both, may be oxidized, and the oxidation may stimulate the conversion of adenosine diphosphate within the mammal into adenosine triphosphate. The osteoblast cells then may convert the adenosine triphosphate into adenosine diphosphate, such that energy is released. Moreover, the osteoblast cells use the energy to synthesize at least Type-I collagen proteins within the mammal, which creates the bone growth.

According to still yet another embodiment of the present invention, a method of performing a medical procedure comprises the steps of positioning at least one suture within an opening formed within or through at least one portion of a bone of a mammal, and dispensing a particular material into the opening. The particular material comprises at least one glyceride and stimulates bone growth within the opening. For example, cyclic adenosine monophosphate regulated lipases may metabolize the particular material after the particular material is positioned within the opening. The at least one glyceride of the particular material and water within the mammal then may be converted into glycerol, fatty acid, and protons, which stimulates a conversion of at least one osteoprogenitor cell into an osteoblast cell. Subsequently, the glycerol or the fatty acid, or both, may be oxidized, and the oxidation may stimulate the conversion of adenosine diphosphate within the mammal into adenosine triphosphate. The osteoblast cells then may convert the adenosine triphosphate into adenosine diphosphate, such that energy is released. Moreover, the osteoblast cells use the energy to synthesize at least Type-I collagen proteins within the mammal, which creates the bone growth.

According to another embodiment of the present invention, a method of performing a medical procedure comprises the steps of forming a mold, and dispensing a liquid, particular material into the mold. The particular material solidifies within the mold and comprises at least one glyceride. Moreover, the mold comprises at least one of a mold for a screw, a mold for a plate, and a mold for a prosthetic member. The method also comprises the steps of removing the solidified, particular material from the mold, and positioning the solidified, particular material one of on a bone of a mammal and within an opening formed within the bone. Moreover, the solidified, particular material stimulates bone growth.

According to another embodiment of the present invention, a method of performing a medical procedure comprises the step of positioning a particular material in the vicinity of a bone of a mammal. Specifically, the particular material comprises at least one glyceride.

18 According to yet another embodiment of the present invention, a method of making a composition used in a medical procedure comprises the step of combining unsaturated vegetable oil ester with adipate to form a first composition. The method also comprises the step of combining the first composition with vegetable oil polyols to form a second composition.

According to still yet another embodiment of the present invention, a particular composition comprises unsaturated vegetable oil ester, adipate, and vegetable oil polyols. Specifically, the unsaturated vegetable oil ester is combined with the adipate to form a further composition, and the further composition is combined with the vegetable oil polyols to form the particular composition.

Other objects, features, and advantages will be apparent to persons of ordinary skill in the art in view of the following detailed description of the invention and the accompanying drawings.

### BRIEF DESCRIPTION OF THE DRAWINGS

For a more complete understanding of the present invention, needs satisfied thereby, and objects, features, and advantages thereof, reference now is made to the following descriptions taken in connection with the accompanying drawings.

FIG. 1a is a Nuclear Magnetic Resonance Spectroscopy for unsaturated vegetable oil ester combined with adipate according to an embodiment of the present invention.

FIG. 1b is a Nuclear Magnetic Resonance Spectroscopy for vegetable oil polyols according to an embodiment of the present invention.

FIG. 2 is a top level flow-chart depicting a first method for performing a medical procedure according to an embodiment of the present invention.

FIG. 3 is a top level flow-chart depicting a second method for performing a medical procedure according to an embodiment of the present invention.

FIG. 4 is a top level flow-chart depicting a third method for performing a medical procedure according to an embodiment of the present invention.

FIG. 5 is a top level flow-chart depicting a fourth method for performing a medical procedure according to an embodiment of the present invention.

FIG. 6 is a top level flow-chart depicting a fifth method for performing a medical procedure according to an embodiment of the present invention.

FIG. 7 is a top level flow-chart depicting a sixth method for performing a medical procedure according to an embodiment of the present invention.

FIG. 8 is a top level flow-chart depicting a seventh method for performing a medical procedure according to an embodiment of the present invention.

FIG. 9 is a top level flow-chart depicting a method for making the composition of FIG. 1.

### DETAILED DESCRIPTION OF PREFERRED EMBODIMENTS

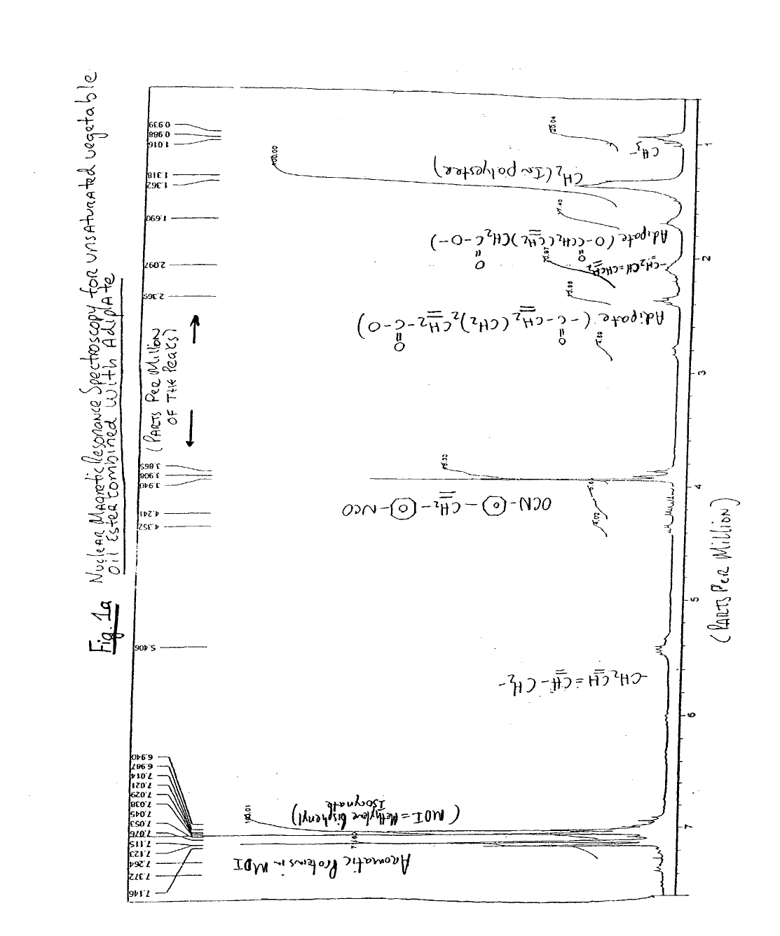

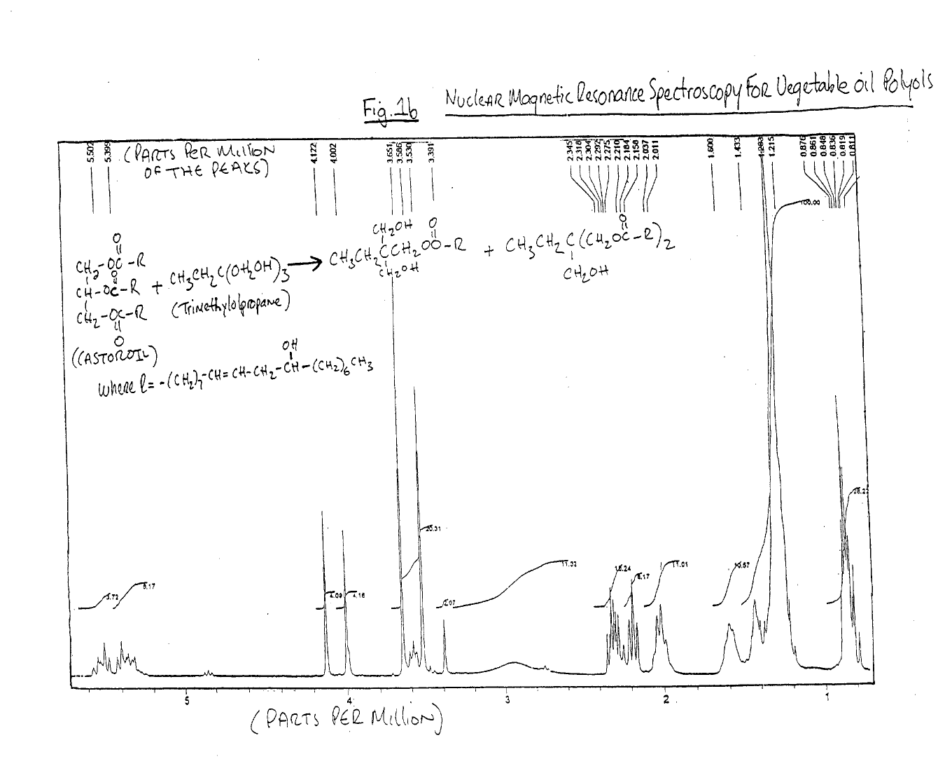

Preferred embodiments of the present invention and their advantages may be understood by referring to FIGS. 1a-10, like numerals being used for like corresponding parts in the various drawings.

Referring to FIGS. 1a and 1b, a final composition for use in medical procedures may comprise unsaturated vegetable oil ester, adipate, and vegetable oil polyols. Specifically, the unsaturated vegetable oil ester may be combined with the adipate to form an initial composition, and the initial composition may be combined with the vegetable oil polyols to form the final composition. Based on the Nuclear Magnetic Resonance Spectroscopy of FIGS. 1a and 1b, those of ordinary skill in the art readily may determine the chemical composition of the initial combination and the vegetable oil polyols. The initial composition may be formed using a known recombinant DNA procedure, a known enzymatic reaction procedure, a known isocyanate based organic synthesis reaction procedure, or the like. Moreover, the final composition may stimulate bone growth when the final composition contacts or is positioned in the vicinity of a bone of a mammal.

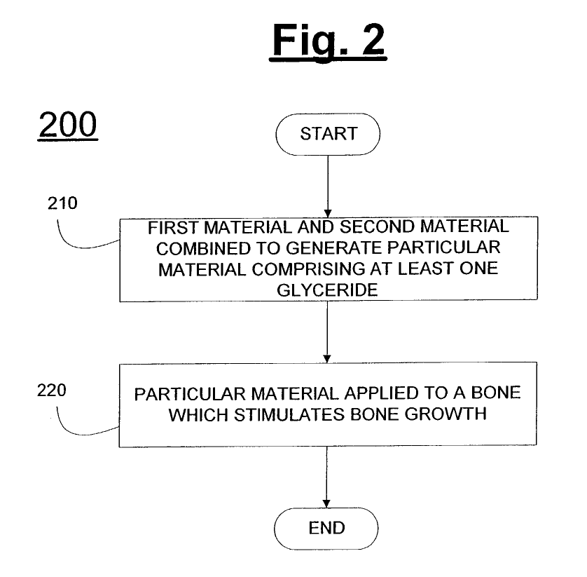

Referring to FIG. 2, a first embodiment of a method 200 for performing a medical procedure, e.g., a non-invasive or an invasive medical procedure, is depicted. In step 210, a first material and a second material may be combined to generate a particular material. The general composition of an exemplary particular material is set forth in FIG. 1. The first material may comprise a first amount of a hydroxyl functional group derived from a vegetable oil, and a second amount of an isocyanate or a di-isocyanate. A ratio between the second amount and the first amount may be greater than a 1:1 molar ratio. Moreover, the second material may comprise a molecule with two or more free hydroxyl functional groups in chain. In an embodiment, at least one of the first material and the second material is partially polymerized. In another embodiment, when the first material is combined with the second material to generate the particular material, the maximum reaction temperature of the particular material is less than a predetermined temperature, e.g., about 45° C. In step 220, the particular material may be applied to a portion of a bone of a mammal. For example, a needle may be inserted through a skin of the mammal the particular material may be dispensed onto a surface the bone, e.g., at a location of a damaged portion of the bone. The particular material may comprise at least one glyceride and also may stimulate bone growth.

While not willing to be bound by a theory, it is believed that cyclic adenosine monophosphate regulated lipases may metabolize the particular material after the particular material is applied to the bone. The at least one glyceride of the particular material and water which naturally is within the mammal then may be converted into glycerol, fatty acid, and protons, which stimulates a conversion of at least one osteoprogenitor cell into an osteoblast cell. Subsequently, the glycerol or the fatty acid, or both, may be oxidized within the mammal, and this oxidation may stimulate the conversion of adenosine diphosphate within the mammal into adenosine triphosphate. The osteoblast cells then may convert the adenosine triphosphate into adenosine diphosphate, such that energy is released. Moreover, the osteoblast cells use the released energy to synthesize at least Type-I collagen proteins within the mammal, which causes or creates the bone growth.

In an embodiment of the present invention, the particular material may be both an adhesive material and a cohesive material. The particular material also may be bacterial static and bactericidal. Further, the particular material may be osteoconductive or osteoinductive. When the particular material is applied to the bone, the particular material may be a liquid, and may conform to a shape the bone. After the particular material is applied to the bone, the particular material may transform into a solid. In another embodiment of the present invention, method 200 also may comprise the step of increasing or decreasing a temperature of the particular material before or after the particular material is applied to the bone. Increasing or decreasing the temperature of the particular material may increase or decrease an amount of time which it takes for the particular material to transform or cure from a liquid to a solid. Moreover, the properties of the particular material may be altered. For example, method 200 also may comprise the step of injecting air, gas, or a fluid inside the particular material, which may change the density of the particular material. In another example, additional components may be added to the particular material to alter the properties of the particular material. For example, calcium, calcium carbonate, calcium hydroxyapatite, calcium phosphate, barium sulfate, hydroxyapatite, salt, and/or bone may be added to the particular material, such that the additional components are suspended within the particular material.

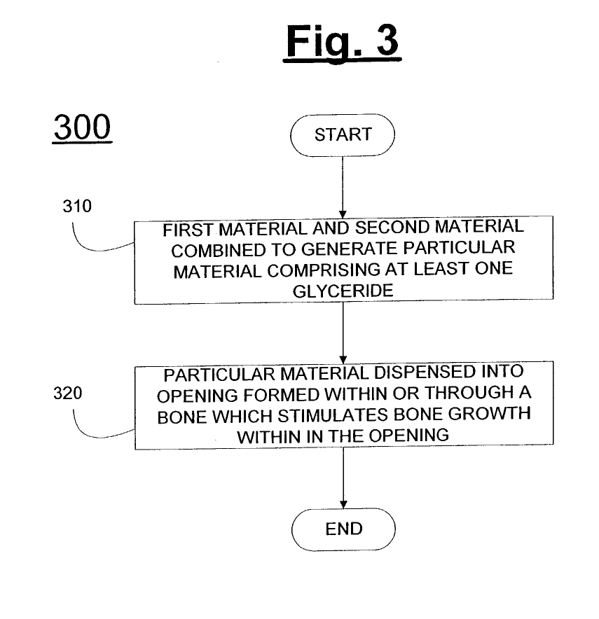

Referring to FIG. 3, a second embodiment of a method 300 for performing a medical procedure, e.g., a non-invasive or an invasive medical procedure, is depicted. The features and advantages of this embodiment of the present invention are substantially similar to the features and advantages of the above-described embodiments of the present invention. Therefore, the similar features and advantages of the above-described embodiments of the present invention are not discussed further with respect to this embodiment of the present invention. In step 310, the first material and the second material may be combined to generate the particular material. In step 320, the particular material is dispensed into an opening formed within or through at least one portion of the bone. Method 300 also may comprise the step of applying pressure to a skin of the mammal which covers the opening in the bone, which may alter the shape of the particular material within the opening.

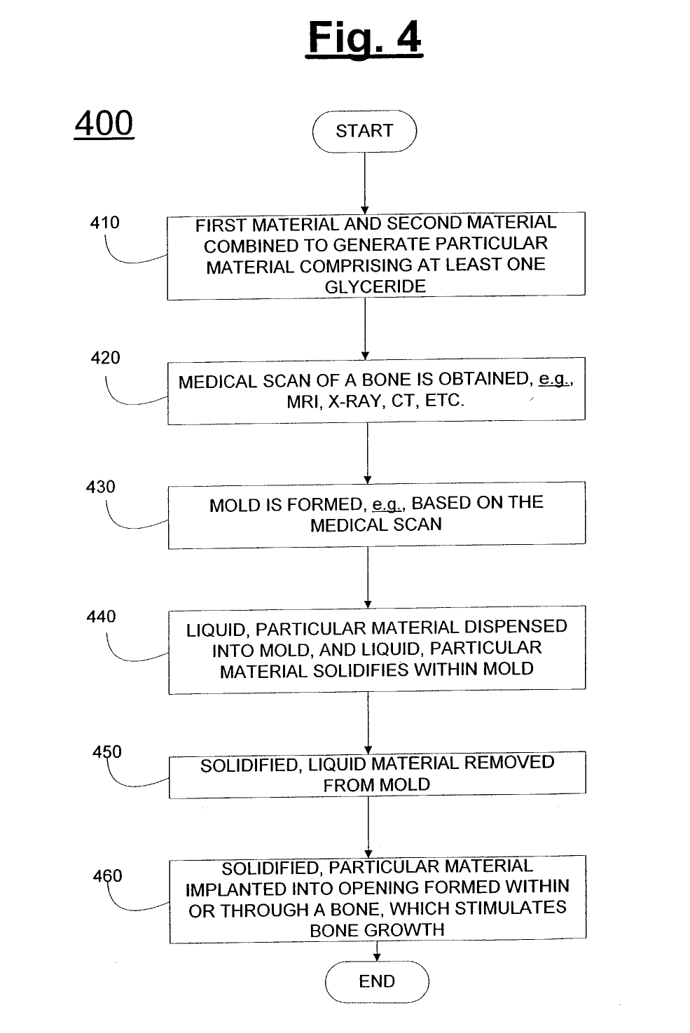

Referring to FIG. 4, a third embodiment of a method 400 for performing a medical procedure, e.g., a non-invasive or an invasive medical procedure, is depicted. The features and advantages of this embodiment of the present invention are substantially similar to the features and advantages of the above-described embodiments of the present invention. Therefore, the similar features and advantages of the above-described embodiments of the present invention are not discussed further with respect to this embodiment of the present invention. In step 410, the first material and the second material may be combined to generate the particular material, and in step 420, a medical scan of the bone may be obtained, e.g., a CT scan, a MRI scan, an X-ray scan, or the like. In step 430, a mold may be formed, e.g., based on the medical scan or based on a generic size for the mold, and in step 440, the liquid, particular material may be dispensed into the mold. The particular material solidifies within the mold. In step 450, the solidified, particular material may removed from the mold, and in step 460, the solidified, particular material may be implanted into an opening formed within or through at least one portion of the bone.

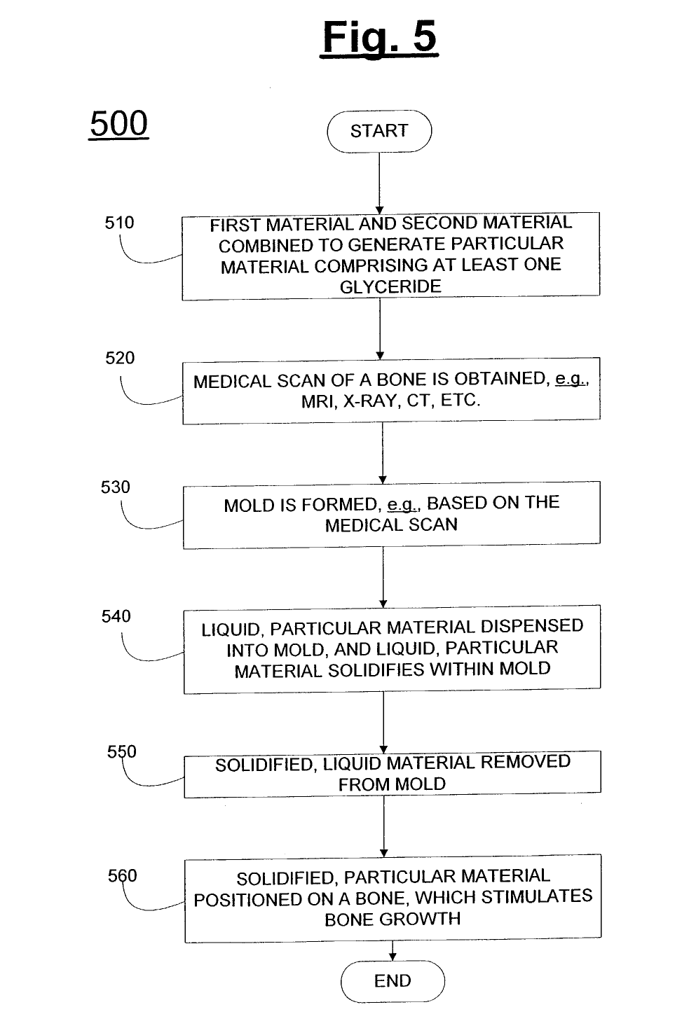

Referring to FIG. 5, a fourth embodiment of a method 500 for performing a medical procedure, e.g., a non-invasive or an invasive medical procedure, is depicted. The features and advantages of this embodiment of the present invention are substantially similar to the features and advantages of the above-described embodiments of the present invention. Therefore, the similar features and advantages of the above-described embodiments of the present invention are not discussed further with respect to this embodiment of the present invention. In step 510, the first material and the second material may be combined to generate the particular material, and in step 520, a medical scan of the bone may be obtained, e.g., a CT scan, a MRI scan, an X-ray scan, or the like. In step 530, a mold may be formed, e.g., based on the medical scan or based on a generic size for the mold, and in step 540, the liquid, particular material may be dispensed into the mold. The particular material solidifies within the mold. In step 550, the solidified, particular material may removed from the mold, and in step 560, the solidified, particular material may be positioned on at least one portion of the bone.

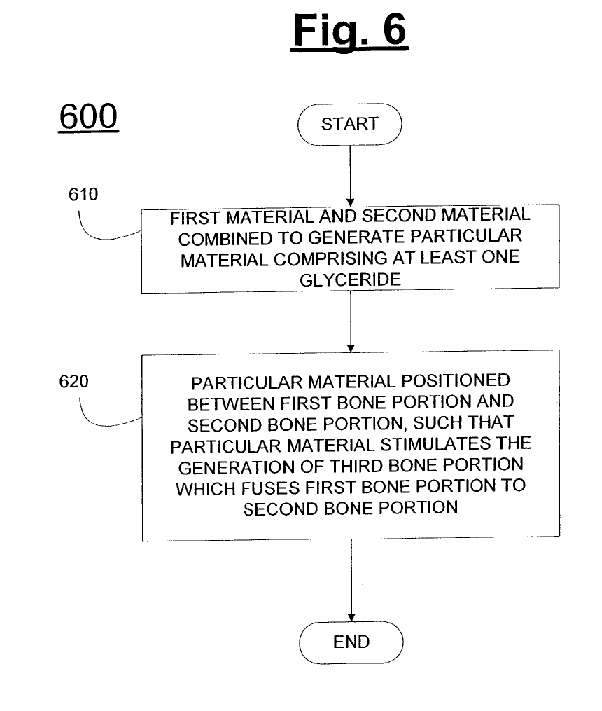

Referring to FIG. 6, a fifth embodiment of a method 600 for performing a medical procedure, e.g., a non-invasive or an invasive medical procedure, is depicted. The features and advantages of this embodiment of the present invention are substantially similar to the features and advantages of the above-described embodiments of the present invention. Therefore, the similar features and advantages of the above-described embodiments of the present invention are not discussed further with respect to this embodiment of the present invention. In step 610, the first material and the second material may be combined to generate the particular material. In step 620, the particular material may be positioned between a first bone portion of the mammal and a second bone portion of the mammal, such that the particular material stimulates the growth of a third bone portion which fuses the first bone portion to the second bone portion. For example, the particular material may be injected into a balloon, and the balloon may be positioned between the first bone portion and the second bone portion. In an embodiment, the balloon may rest on tissue of the mammal, and the tissue may degrade the balloon before the particular material solidifies. Moreover, the same bone within the mammal may comprise each of the first bone portion and the second bone portion, or a first bone may comprise the first bone portion and a second bone may comprise the second bone portion. For example, the particular bone may be a first vertebra of a spine of the mammal and the second bone may be a second vertebra of the spine.

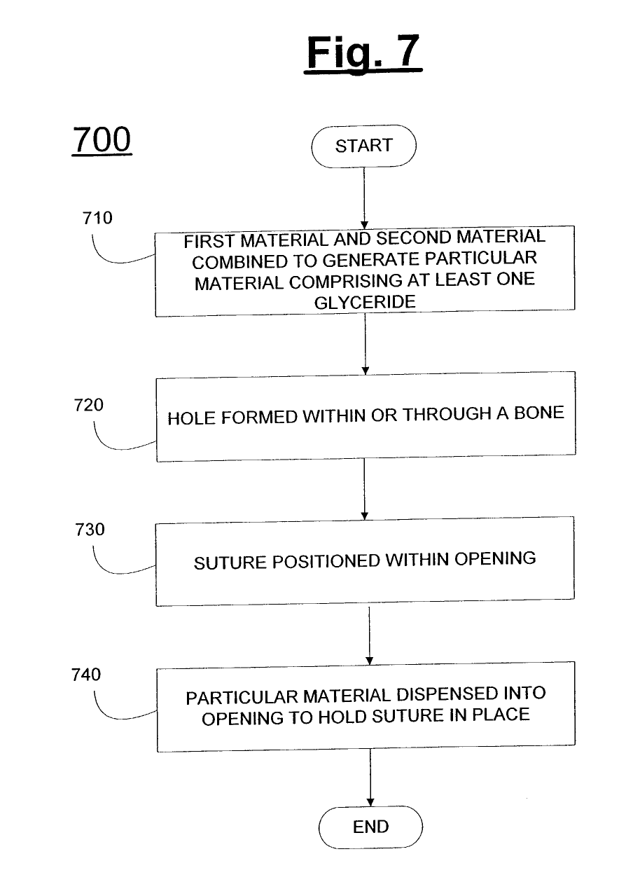

Referring to FIG. 7, a sixth embodiment of a method 700 for performing a medical procedure, e.g., a non-invasive or an invasive medical procedure, is depicted. The features and advantages of this embodiment of the present invention are substantially similar to the features and advantages of the above-described embodiments of the present invention. Therefore, the similar features and advantages of the above-described embodiments of the present invention are not discussed further with respect to this embodiment of the present invention. In step 710, the first material and the second material may be combined to generate the particular material. In step 720, a hole may be formed, e.g., drilled, within or through the bone. In step 730, at least one suture may be positioned within the opening formed within or through the bone. For example, a fluid or a powder which prevents the suture from adhering to the particular material may be applied to the suture, and then the suture may be dispensed in the opening. In step 740, the particular material may be dispensed into the opening to prevent the suture from falling out of the opening.

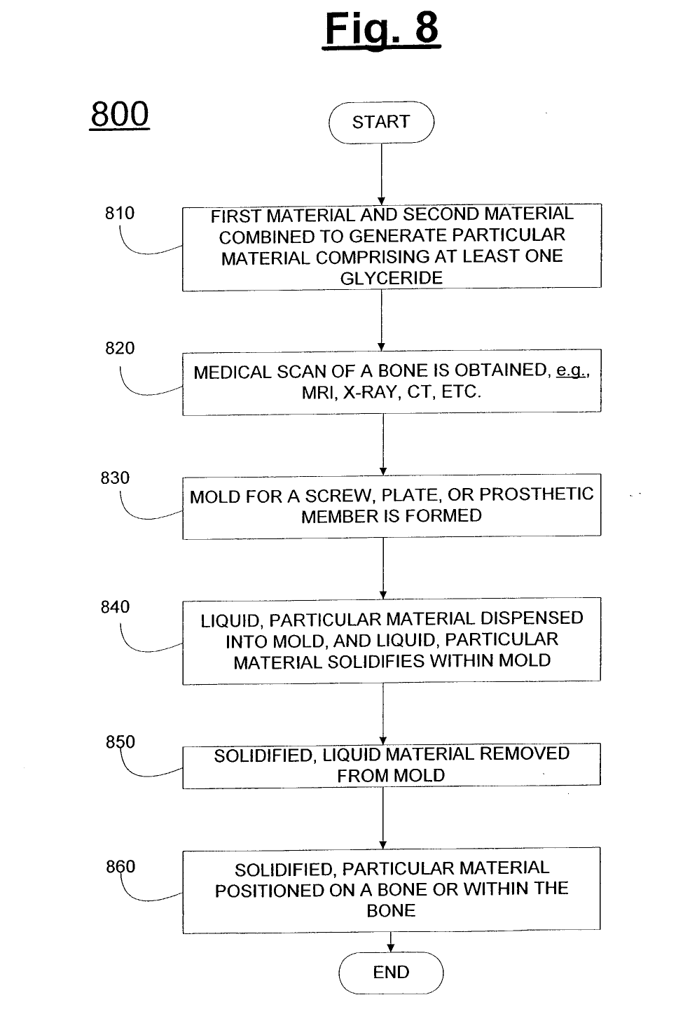

Referring to FIG. 8, an seventh embodiment of a method 800 for performing a medical procedure, e.g., a non-invasive or an invasive medical procedure, is depicted. The features and advantages of this embodiment of the present invention are substantially similar to the features and advantages of the above-described embodiments of the present invention. Therefore, the similar features and advantages of the above-described embodiments of the present invention are not discussed further with respect to this embodiment of the present invention. In step 810, the first material and the second material may be combined to generate the particular material, and in step 820, a medical scan of the bone may be obtained, e.g., a CT scan, a MRI scan, an X-ray scan, or the like. In step 830, a mold may be formed, and the mold may comprise a mold for a screw, a mold for a plate, a mold for a prosthetic member, or the like. In step 840, the liquid, particular material may be dispensed into the mold. The particular material solidifies within the mold. In step 850, the solidified, particular material may removed from the mold, and in step 860, the solidified, particular material may be positioned on a bone of a mammal or within an opening formed within the bone.

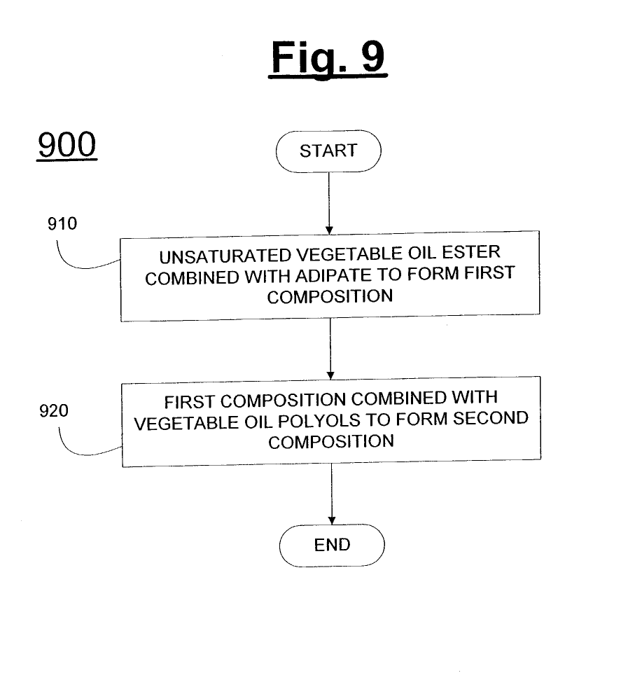

Referring to FIG. 9, a method 900 of making a composition used in a medical procedure is depicted. In step 910, unsaturated vegetable oil ester (which is a glyceride) may be combined with adipate to form a first composition, and in step 920, the first composition may be combined with vegetable oil polyols to form a second composition.

While the invention has been described in connection with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein. It is intended that the specification and the described examples are considered as exemplary only, with the true scope and spirit of the invention indicated by the following claims.

What is claimed is:

1. A method of performing a medical procedure, comprising the step of applying a particular material to at least one portion of a bone of a mammal, wherein the particular material comprises at least one glyceride and stimulates bone growth.

2. The method of claim 1, wherein the particular material stimulates the conversion of at least one osteoprogenitor cell into an osteoblast cell.

3. The method of claim 1, wherein at least one portion of the particular material is metabolized by cyclic adenosine monophosphate regulated lipases after the particular material is applied to the at least one portion of the bone.

4. The method of claim 3, wherein when the cyclic adenosine monophosphate regulated lipase metabolizes the at least one portion of the particular material, the at least one glyceride of the particular material and water within the mammal is converted into glycerol, fatty acid, and protons, which stimulates a conversion of at least one osteoprogenitor cell into an osteoblast cell.

5. The method of claim 4, wherein after the conversion of the at least one glyceride of the particular material and the water within the mammal into glycerol, fatty acid, and protons, at least one of the glycerol and the fatty acid is oxidized, and the oxidation stimulates the conversion of adenosine diphosphate within the mammal into adenosine triphosphate.

6. The method of claim 5, wherein after the conversion of the adenosine diphosphate within the mammal into adenosine triphosphate, the osteoblast cells convert the adenosine triphosphate into adenosine diphosphate, such that energy is released and the osteoblast cells use the energy to synthesize at least Type-I collagen proteins within the mammal.

7. The method of claim 6, wherein the synthesis of the Type-I collagen proteins create bone.

8. The method of claim 1, wherein the particular material is each of adhesive and cohesive.

9. The method of claim 1, wherein the particular material is each of bacterial static and bactericidal.

10. The method of claim 1, wherein the at least one glyceride comprises a first glyceride, a second glyceride, and a third glyceride.

11. The method of claim 10, wherein the second glyceride is different than the first glyceride, and the third glyceride is different than each of the first glyceride and the second glyceride.

12. The method of claim 1, further comprising the step of combining a first material and a second material to generate the particular material, wherein the first material comprises:
a first amount of a hydroxyl functional group derived from a vegetable oil; and
a second amount of an isocyanate or a di-isocyanate, wherein a ratio between the second amount and the first amount is greater than a 1:1 molar ratio, and wherein the second material comprises a molecule with two or more free hydroxyl functional groups in chain.

13. The method of claim 12, wherein the combining step is performed before the applying step.

14. The method of claim 12, wherein at least one of the first material and the second material is partially polymerized.

15. The method of claim 12, wherein when the first material is combined with the second material to generate the particular material, the maximum reaction temperature of the particular material is less than a predetermined temperature.

16. The method of claim 15, wherein the predetermined temperature is about 45° C.

17. The method of claim 1, wherein the particular material conforms to a shape of the at least one portion of the bone when the particular material is applied to the at least one portion of the bone.

18. The method of claim 1, wherein the particular material is a liquid and transforms into a solid after the applying step.

19. The method claim 1, further comprising the step of increasing a temperature of the particular material before or after the applying step.

20. The method claim 1, further comprising the step of decreasing a temperature of the particular material before or after the applying step.

21. The method of claim 1, wherein the particular material is osteoconductive.

22. The method of claim 1, wherein the particular material is osteoinductive.

23. The method of claim 1, further comprising the step of injecting at least one of air, gas, and a fluid inside the particular material.

24. The method of claim 23, wherein the injecting step is performed after the applying step.

25. The method of claim 24, wherein the combining step is performed before the applying step.

26. The method of claim 1, wherein the particular material further comprises at least one of calcium, calcium carbonate, calcium hydroxyapatite, calcium phosphate, barium sulfate, hydroxyapatite salt, and bone suspended within the particular material.

27. The method of claim 1, wherein the particular material is applied to the at least one portion of the bone by inserting a needle through a skin of the mammal.

28. The method of claim 1, wherein the medical procedure is a surgical procedure.

29. A method of performing a medical procedure, comprising the step of dispensing a particular material into an opening formed within or through at least one portion of a bone of a mammal, wherein the particular material comprises at least one glyceride and stimulates bone growth within the opening.

30. The method of claim 29, wherein the particular material stimulates the conversion of at least one osteoprogenitor cell into an osteoblast cell.

31. The method of claim 29, wherein at least one portion of the particular material is metabolized by cyclic adenosine monophosphate regulated lipases after the particular material is dispensed within the opening.

32. The method of claim 31, wherein when the cyclic adenosine monophosphate regulated lipase metabolizes the at least one portion of the particular material, the at least one glyceride of the particular material and water within the mammal is converted into glycerol, fatty acid, and protons, which stimulates a conversion of at least one osteoprogenitor cell into an osteoblast cell.

33. The method of claim 32, wherein after the conversion of the at least one glyceride of the particular material and the water within the mammal into glycerol, fatty acid, and protons, at least one of the glycerol and the fatty acid is oxidized, and the oxidation stimulates the conversion of adenosine diphosphate within the mammal into adenosine triphosphate.

34. The method of claim 33, wherein after the conversion of the adenosine diphosphate within the mammal into adenosine triphosphate, the osteoblast cells convert the adenosine triphosphate into adenosine diphosphate, such that energy is released and the osteoblast cells use the energy to synthesize at least Type-I collagen proteins within the mammal.

35. The method of claim 34, wherein the synthesis of the Type-I collagen proteins create the bone growth.

36. The method of claim 29, wherein the particular material is each of adhesive and cohesive.

37. The method of claim 29, wherein the particular material is each of bacterial static and bactericidal.

38. The method of claim 29, wherein the at least one glyceride comprises a first glyceride, a second glyceride, and a third glyceride.

39. The method of claim 38, wherein the second glyceride is different than the first glyceride, and the third glyceride is different than each of the first glyceride and the second glyceride.

40. The method of claim 29, further comprising the step of combining a first material and a second material to generate the particular material, wherein the first material comprises:
a first amount of a hydroxyl functional group derived from a vegetable oil; and
a second amount of an isocyanate or a di-isocyanate, wherein a ratio between the second amount and the first amount is greater than a 1:1 molar ratio, and wherein the second material comprises a molecule with two or more free hydroxyl functional groups in chain.

41. The method of claim 40, wherein the combining step is performed before the dispensing step.

42. The method of claim 40, wherein at least one of the first material and the second material is partially polymerized.

43. The method of claim 40, wherein when the first material is combined with the second material to generate the particular material, the maximum reaction temperature of the particular material is less than a predetermined temperature.

44. The method of claim 43, wherein the predetermined temperature is about 45° C.

45. The method of claim 29, wherein the particular material conforms to a shape of the opening when the particular material is dispensed within the opening.

46. The method of claim 29, wherein the particular material is a liquid and transforms into a solid after the dispensing step.

47. The method claim 29, further comprising the step increasing a temperature of the particular material before or after the dispensing step.

48. The method claim 29, further comprising the step decreasing a temperature of the particular material before or after the dispensing step.

49. The method of claim 29, wherein the particular material is osteoconductive.

50. The method of claim 29, wherein the particular material is osteoinductive.

51. The method of claim 29, further comprising the step of injecting at least one of air, gas, and a fluid inside the particular material.

52. The method of claim 23, wherein the injecting step is performed after the dispensing step.

53. The method of claim 24, wherein the combining step is performed before the dispensing step.

54. The method of claim 29, wherein the particular material further comprises at least one of calcium, calcium carbonate, calcium hydroxyapatite, calcium phosphate, barium sulfate, hydroxyapatite salt, and bone suspended within the particular material.

55. The method of claim 29, wherein the particular material is dispensed within the opening by inserting a needle through a skin of the mammal.

56. The method of claim 29, further comprising the step of applying a pressure to a skin of the mammal after a completion of the dispensing step, wherein applying the pressure altars a shape of the particular material within the opening.

57. The method of claim 29, wherein the medical procedure is a surgical procedure.

58. A method of performing a medical procedure, comprising the steps of:
forming a mold;
dispensing a liquid, particular material into the mold, wherein the particular material solidifies within the mold and comprises at least one glyceride;
removing the solidified, particular material from the mold; and
implanting the solidified, particular material into an opening formed within or through at least one portion of a bone of a mammal, wherein the implanted, particular material stimulates bone growth.

59. The method of claim 58, further comprising the step of obtaining a medical scan of the at least one portion of the bone, wherein the mold is formed based on the medical scan.

60. The method of claim 59, wherein the medical scan is at least one of an X-ray, a Magnetic Resonance Imaging, and a Computer Tomography.

61. The method of claim 58, wherein at least one portion of the solidified, particular material is metabolized by cyclic adenosine monophosphate regulated lipases after the solidified, particular material is implanted within the opening.

62. The method of claim 61, wherein when the cyclic adenosine monophosphate regulated lipase metabolizes the at least one portion of the solidified, particular material, the at least one glyceride of the solidified, particular material and water within the mammal is converted into glycerol, fatty acid, and protons, which stimulates a conversion of at least one osteoprogenitor cell into an osteoblast cell.

63. The method of claim 62, wherein after the conversion of the at least one glyceride of the particular material and the water within the mammal into glycerol, fatty acid, and protons, at least one of the glycerol and the fatty acid is oxidized, and the oxidation stimulates the conversion of adenosine diphosphate within the mammal into adenosine triphosphate.

64. The method of claim 63, wherein after the conversion of the adenosine diphosphate within the mammal into adenosine triphosphate, the osteoblast cells convert the adenosine triphosphate into adenosine diphosphate, such that energy is released and the osteoblast cells use the energy to synthesize at least Type-I collagen proteins within the mammal.

65. The method of claim 64, wherein the synthesis of the Type-I collagen proteins create the bone growth.

66. The method of claim 58, further comprising the step of combining a first material and a second material to generate the liquid, particular material, wherein the first material comprises:
a first amount of a hydroxyl functional group derived from a vegetable oil; and
a second amount of an isocyanate or a di-isocyanate, wherein a ratio between the second amount and the first amount is greater than a 1:1 molar ratio, and wherein the second material comprises a molecule with two or more free hydroxyl functional groups in chain.

67. The method of claim 66, wherein the combining step is performed before the dispensing step.

68. The method of claim 66, wherein at least one of the first material and the second material is partially polymerized.

69. The method of claim 58, wherein the medical procedure is a surgical procedure.

70. A method of performing a medical procedure, comprising the steps of:
forming a mold;
dispensing a liquid, particular material into the mold, wherein the particular material solidifies within the mold and comprises at least one glyceride;
removing the solidified, particular material from the mold; and
positioning the solidified, particular material on at least one portion of a bone of a mammal, wherein the positioned, particular material stimulates bone growth.

71. The method of claim 70, further comprising the step of obtaining a medical scan of the at least one portion of the bone, wherein the mold is formed based on the medical scan.

72. The method of claim 71, wherein the medical scan is at least one of an X-ray, a Magnetic Resonance Imaging, and a Computer Tomography.

73. The method of claim 70, wherein at least one portion of the solidified, particular material is metabolized by cyclic adenosine monophosphate regulated lipases after the solidified, particular material is positioned on the at least one portion of the bone.

74. The method of claim 73, wherein when the cyclic adenosine monophosphate regulated lipase metabolizes the at least one portion of the solidified, particular material, the at least one glyceride of the solidified, particular material and water within the mammal is converted into glycerol, fatty acid, and protons, which stimulates a conversion of at least one osteoprogenitor cell into an osteoblast cell.

75. The method of claim 74, wherein after the conversion of the at least one glyceride of the particular material and the water within the mammal into glycerol, fatty acid, and protons, at least one of the glycerol and the fatty acid is oxidized, and the oxidation stimulates the conversion of adenosine diphosphate within the mammal into adenosine triphosphate.

76. The method of claim 75, wherein after the conversion of the adenosine diphosphate within the mammal into adenosine triphosphate, the osteoblast cells convert the adenosine triphosphate into adenosine diphosphate, such that energy is released and the osteoblast cells use the energy to synthesize at least Type-I collagen proteins within the mammal.

77. The method of claim 76, wherein the synthesis of the Type-I collagen proteins create the bone growth.

78. The method of claim 70, further comprising the step of combining a first material and a second material to generate the liquid, particular material, wherein the first material comprises:
a first amount of a hydroxyl functional group derived from a vegetable oil; and
a second amount of an isocyanate or a di-isocyanate, wherein a ratio between the second amount and the first amount is greater than a 1:1 molar ratio, and wherein the second material comprises a molecule with two or more free hydroxyl functional groups in chain.

79. The method of claim 78, wherein the combining step is performed before the dispensing step.

80. The method of claim 78, wherein at least one of the first material and the second material is partially polymerized.

81. The method of claim 70, wherein the medical procedure is a surgical procedure.

82. A method of performing a medical procedure, comprising the step of injecting a particular material into a bone of a mammal, wherein the particular material comprises at least one glyceride and stimulates bone growth within the bone.

83. The method of claim 82, wherein the particular material is injected into the bone using a needle which is inserted into the bone to form an opening within the bone.

84. The method of claim 83, wherein the needle is inserted through a skin of the mammal before the needle is inserted into the bone.

85. The method of claim 82, wherein at least one portion of the particular material is metabolized by cyclic adenosine monophosphate regulated lipases after the particular material is injected into the bone.

86. The method of claim 85, wherein when the cyclic adenosine monophosphate regulated lipase metabolizes the at least one portion of the particular material, the at least one glyceride of the solidified, particular material and water within the mammal is converted into glycerol, fatty acid, and protons, which stimulates a conversion of at least one osteoprogenitor cell into an osteoblast cell.

87. The method of claim 86, wherein after the conversion of the at least one glyceride of the particular material and the water within the mammal into glycerol, fatty acid, and protons, at least one of the glycerol and the fatty acid is oxidized, and the oxidation stimulates the conversion of adenosine diphosphate within the mammal into adenosine triphosphate.

88. The method of claim 87, wherein after the conversion of the adenosine diphosphate within the mammal into adenosine triphosphate, the osteoblast cells convert the adenosine triphosphate into adenosine diphosphate, such that energy is released and the osteoblast cells use the energy to synthesize at least Type-I collagen proteins within the mammal.

89. The method of claim 88, wherein the synthesis of the Type-I collagen proteins create the bone growth.

90. The method of claim 82, further comprising the step of combining a first material and a second material to generate the particular material, wherein the first material comprises:
a first amount of a hydroxyl functional group derived from a vegetable oil; and
a second amount of an isocyanate or a di-isocyanate, wherein a ratio between the second amount and the first amount is greater than a 1:1 molar ratio, and wherein the second material comprises a molecule with two or more free hydroxyl functional groups in chain.

91. The method of claim 90, wherein the combining step is performed before the injecting step.

92. The method of claim 90, wherein at least one of the first material and the second material is partially polymerized.

93. The method of claim 82, wherein the medical procedure is a surgical procedure.

94. A method of performing a medical procedure, comprising the step of positioning a particular material between a first bone portion of a mammal and a second bone portion of the mammal, wherein the particular material comprises at least one glyceride and stimulates a growth of a third bone portion which fuses the first bone portion to the second bone portion.

95. The method of claim 94, wherein the positioning step comprises the substeps of:
injecting the particular material into a balloon; and
positioning the balloon between the first bone portion and the second bone portion.

96. The method of claim 94, wherein a particular bone comprises each of the first bone portion and the second bone portion.

97. The method of claim 94, wherein a particular bone comprises the first bone portion and a further bone comprises the second bone portion.

98. The method of claim 97, wherein the particular bone is a first vertebra of a spine of the mammal and the further bone is a second vertebra of the spine.

99. The method of claim 94, wherein at least one portion of the particular material is metabolized by cyclic adenosine monophosphate regulated lipases after the particular material is positioned between the first bone portion and the second bone portion.

100. The method of claim 99, wherein when the cyclic adenosine monophosphate regulated lipase metabolizes the at least one portion of the particular material, the at least one glyceride of the particular material and water within the mammal is converted into glycerol, fatty acid, and protons, which stimulates a conversion of at least one osteoprogenitor cell into an osteoblast cell.

101. The method of claim 100, wherein after the conversion of the at least one glyceride of the particular material and the water within the mammal into glycerol, fatty acid, and protons, at least one of the glycerol and the fatty acid is oxidized, and the oxidation stimulates the conversion of adenosine diphosphate within the mammal into adenosine triphosphate.

102. The method of claim 101, wherein after the conversion of the adenosine diphosphate within the mammal into adenosine triphosphate, the osteoblast cells convert the adenosine triphosphate into adenosine diphosphate, such that energy is released and the osteoblast cells use the energy to synthesize at least Type-I collagen proteins within the mammal.

103. The method of claim 102, wherein the synthesis of the Type-I collagen proteins create the growth of the third bone portion.

104. The method of claim 94, further comprising the step of combining a first material and a second material to generate the particular material, wherein the first material comprises:
a first amount of a hydroxyl functional group derived from a vegetable oil; and
a second amount of an isocyanate or a di-isocyanate, wherein a ratio between the second amount and the first amount is greater than a 1:1 molar ratio, and wherein the second material comprises a molecule with two or more free hydroxyl functional groups in chain.

105. The method of claim 104, wherein the combining step is performed before the positioning step.

106. The method of claim 104, wherein at least one of the first material and the second material is partially polymerized.

107. The method of claim 94, wherein the medical procedure is a surgical procedure.

108. A method of performing a medical procedure, comprising the steps of:
positioning at least one suture within an opening formed within or through at least one portion of a bone of a mammal; and
dispensing a particular material into the opening, wherein the particular material comprises at least one glyceride and stimulates bone growth within the opening.

109. The method of claim 108, further comprising the steps of:
forming the opening before positioning the at least one suture within the opening; and
applying one of a liquid and a powder to the suture.

110. The method of claim 108, wherein at least one portion of the particular material is metabolized by cyclic adenosine monophosphate regulated lipases after the particular material is dispensed into the opening.

111. The method of claim 110, wherein when the cyclic adenosine monophosphate regulated lipase metabolizes the at least one portion of the particular material, the at least one glyceride of the particular material and water within the mammal is converted into glycerol, fatty acid, and protons, which stimulates a conversion of at least one osteoprogenitor cell into an osteoblast cell.

112. The method of claim 111, wherein after the conversion of the at least one glyceride of the particular material and the water within the mammal into glycerol, fatty acid, and protons, at least one of the glycerol and the fatty acid is oxidized, and the oxidation stimulates the conversion of adenosine diphosphate within the mammal into adenosine triphosphate.

113. The method of claim 112, wherein after the conversion of the adenosine diphosphate within the mammal into adenosine triphosphate, the osteoblast cells convert the adenosine triphosphate into adenosine diphosphate, such that energy is released and the osteoblast cells use the energy to synthesize at least Type-I collagen proteins within the mammal.

114. The method of claim 113, wherein the synthesis of the Type-I collagen proteins create the bone growth.

115. The method of claim 108, further comprising the step of combining a first material and a second material to generate the particular material, wherein the first material comprises:
a first amount of a hydroxyl functional group derived from a vegetable oil; and
a second amount of an isocyanate or a di-isocyanate, wherein a ratio between the second amount and the first amount is greater than a 1:1 molar ratio, and wherein the second material comprises a molecule with two or more free hydroxyl functional groups in chain.

116. The method of claim 115, wherein the combining step is performed before the dispensing step.

117. The method of claim 115, wherein at least one of the first material and the second material is partially polymerized.

118. The method of claim 108, wherein the medical procedure is a surgical procedure.

119. A method of performing a medical procedure, comprising the steps of:
forming a mold;
dispensing a liquid, particular material into the mold, wherein the particular material solidifies within the mold and comprises at least one glyceride, and wherein the mold comprises at least one of a mold for a screw, a mold for a plate, and a mold for a prosthetic member;
removing the solidified, particular material from the mold; and
positioning the solidified, particular material one of on a bone of a mammal and within an opening formed within the bone, wherein the solidified, particular material stimulates bone growth.

120. A method of performing a medical procedure, comprising the step of positioning a particular material in the vicinity of a bone of a mammal, wherein the particular material comprises at least one glyceride.

121. The method of claim 120, wherein the positioning step comprises the substep of applying the particular material to at least one portion of the bone.

122. The method claim 121, wherein the positioning step comprises the substep of dispensing the particular material in an opening formed within or through the bone.

123. The method claim 121, wherein the positioning step comprises the substep of injecting the particular material into the bone.

124. A method of making a composition used in a medical procedure, comprising the steps of:
combining unsaturated vegetable oil ester with adipate to form a first composition; and
combining the first composition with vegetable oil polyols to form a second composition.

125. The method of claim 124, wherein the second composition stimulates bone growth when the second composition contacts or is positioned in the vicinity of a bone of a mammal.

126. The method of claim 124, wherein the first composition is formed using a recombinant DNA procedure

127. The method of claim 124, wherein the first composition is formed using an enzymatic reaction procedure.

128. The method of claim 124, wherein the first composition is formed using an isocyanate based organic synthesis reaction procedure.

129. A particular composition, comprising:
unsaturated vegetable oil ester;
adipate; and
vegetable oil polyols, wherein the unsaturated vegetable oil ester is combined with the adipate to form a further composition, and the further composition is combined with the vegetable oil polyols to form the particular composition.

130. The particular composition of claim 129, wherein the particular composition stimulates bone growth when the particular composition contacts or is positioned in the vicinity of a bone of a mammal.

131. The particular composition of claim 129, wherein the particular composition uniformly expands a predetermined amount of time after the further composition is combined with the vegetable oil polyols.
